(19) United States
(12) Patent Application Publication
(10) Pub. No.: US 2008/0255037 A1
(43) Pub. Date: Oct. 16, 2008

(54) Endoparasiticidal Compositions

(75) Inventors: Ventaka-Rangarao Kanikanti, Leverkusen (DE); Michael Traubel, Koln (DE)

Correspondence Address: BAYER HEALTHCARE LLC, P.O.BOX 390, SHAWNEE MISSION, KS, 66201, US

(73) Assignee: Bayer Healthcare AG, Leverkusen (DE)

(21) Appl. No.: 11/908,309

(22) PCT Filed: Feb. 27, 2006
(86) PCT No.: PCT/EP06/01759

§ 371 Date: Feb. 8, 2008

(30) Foreign Application Priority Data
Mar. 11, 2005 (DE) 10 2005 011 779.1

(51) Int. Cl. A61K 38/12; A61P 33/00

(52) U.S. Cl. 514/11

(57) ABSTRACT

The present invention relates to compositions for external application which comprise emodepside and praziquantel or epsiprantel and 1,2-isopropylideneglycerol, to their preparation and to their use for controlling endoparasites.

The present invention relates to compositions for external application which comprise emodepside and praziquantel or epsiprantel and 1,2-isopropylideneglycerol, to their preparation and to their use for controlling endoparasites.

The anthelmintically active compound praziquantel (U.S. Pat. No. 4,001,411) and the structurally related active compound epsiprantel (U.S. Pat. No. 4,661,489) are usually administered orally, see, for example, DE-A-199 41 024, WO 98/03157, US 2002/0081292 A1 and WO 97/25976. In the case of topical application of endoparasiticides, the active compound has to pass into the bloodstream through the skin in order to reach the endoparasites in question. Since praziquantel and epsiprantel are not particularly suitable for transdermal application, the topical transdermal application form is, owing to the difficulties to be expected, uncommon for these active compounds. A composition for the dermal treatment of helmintic diseases using praziquantel is described in EP-A-267 404. However, the application of this composition is limited to cats where effective transdermal application is generally considerably easier to achieve than, for example, in the case of dogs.

WO 01/60380 (Phoenix Scientific, Inc.) discloses parasiticidal formulations for injection or for pour-on application, which formulations may comprise a pyrrolidone solvent, a further solvent and a parasiticidally active compound. The extensive list of active compounds mentions, inter alia, praziquantel.

EP-A-1 308 163 (Wyeth) discloses endoparasiticidal compositions in the form of gels comprising moxidectin, praziquantel, benzyl alcohol, ethanol, colloidal silica, a surfactant and an oil.

WO 95/23590 (Bomac Laboratories) discloses a complicated process for preparing anthelmintic compositions for dermal application. The compositions comprise a carrier, an emulsifier, an oil and a diluent. Suitable active compounds are especially benzimidazoles, but macrocyclic lactones and praziquantel are also mentioned, inter alia.

WO 02/094288 describes a veterinary pharmaceutical composition which comprises an avermectin oxime derivative, in particular selamectin, in combination with praziquantel. Proposed routes of administration include topical application; corresponding formulations comprise a di(C₂₋₄-glycol) mono(C₁₋₄-alkyl)ether and, if appropriate, a skin-friendly solvent.

The cyclic depsipeptide PF1022 and its action against endoparasites is known from EP-A 382 173.

Further cyclic depsipeptides and their endoparasiticidal action are subject-matter of the German patent applications EP-A 626 376; EP-A 626 375; EP-A 644 883.

The anthelmintically active cyclic depsipeptide emodepside is known from WO 93/19053.

Endoparasiticidal compositions comprising praziquantel or epsiprantel and cyclic depsipeptides are described in EP 662 326.

WO 96/38165 provides endoparasiticidal compositions comprising avermectins, ivermectins, milbemycins in combination with cyclic depsipeptides and also, if appropriate, praziquantel or epsiprantel.

Compositions for dermal application comprising depsipeptides, such as, for example, emodepside, are described, inter alia, in WO 01/62268 and in our application WO 05/055973.

However, the level of activity and/or duration of action of the compositions of the prior art is, in particular in the case of certain hosts, against certain organisms and/or at low application concentrations, not entirely satisfactory in all areas of use.

Because of the wide variety of requirements to be met by modern pharmaceuticals, for example concerning level of activity (for example plasma concentration of the active compound), duration of action, spectrum of action, range of applications, toxicity, combination of active compounds, combination with formulation auxiliaries, and because of the possible occurrence of resistance, the development of novel pharmaceuticals cannot ever be regarded as complete, and there is a continuing great need for novel compositions which have advantages, at least in some aspects, over the known compositions.

To enable the pet owner to apply endoparasiticidally active compounds in a manner which is as simple as possible, it is furthermore desirable to provide an externally applicable composition, external application in the context of the present application generally meaning application to the skin or the coat of animals.

As is known from the literature, it is extremely difficult for molecules having molecular weights>1000 u to penetrate the skin when applied topically. Particularly poor is the penetration of peptides or proteins having relatively large molecular weights (Cevc et al., Advanced Drug Delivery Reviews 18 (1996) 349-378; Bauer et al. Pharmazeutische Technologie, 1993, p. 364, Thieme Verlag; Gurny et al. Dermal and Transdermal Drug Delivery, 1993, p. 131, Wissenschaftliche Verlagsgesellschaft). However, in the case of endoparasiticidally active compounds, penetration is a precondition, since the active compounds are intended to act against endoparasites, for example in the gastrointestinal tract.

Although a few publications of the prior art propose the topical application of praziquantel and/or cyclic depsipeptides, it is known to the person skilled in the art that these active compounds are not particularly suitable for this purpose and that, as a consequence, the known formulations are not entirely satisfactory, in particular in the case of, for example, so-called dose-driving worms, such as the whipworm Trichuris vulpis and/or the tapeworm Taenia canis.

An endoparasiticidal composition has now been found which, when applied externally, shows very good action against a broad spectrum of endoparasites and at the same time has good stability.

The present invention provides:

compositions comprising, as active compounds,
emodepside and
praziquantel or epsiprantel
   and, as solvent, 1,2-isopropylideneglycerol, where the water content of the compositions is at most 1% by weight.

The invention furthermore provides a process for preparing such compositions in which the active compounds are mixed with the solvents and, if appropriate, further auxiliaries.

The INN emodepside denotes the compound having the systematic name: cyclo[(R)-lactoyl-N-methyl-1-leucyl-(R)-3-(p-morpholinophenyl)lactoyl-N-methyl-1-leucyl-(R)-lactoyl-N-methyl-1-leucyl-(R)-3-(p-morpholinophenyl)lactoyl-N-methyl-1-leucyl]. Emodepside has been described in WO 93/19053 and has the formula below:

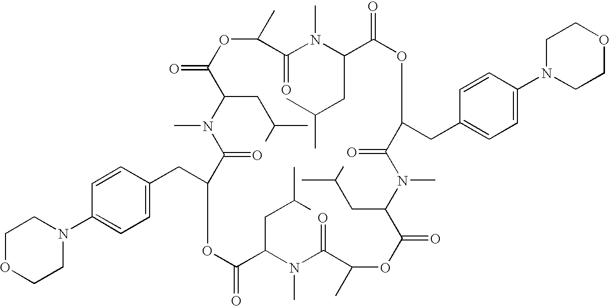

Praziquantel and epsiprantel have been known for a long time as active compounds against endoparasites (see, for example, U.S. Pat. No. 4,661,489 for epsiprantel and U.S. Pat. No. 4,001,411 for praziquantel).

Praziquantel-containing products are commercially available, for example under the name Droncit®. In the context of the present invention, the use of praziquantel is preferred.

According to the invention, it is possible to use both pure stereoisomers and mixtures of stereoisomers. If chemically possible, it is also feasible to use pharmaceutically acceptable salts. Furthermore, it is also possible to use prodrugs of the active compounds in the compositions, the actual active compounds being released after application of the prodrugs from the latter.

The compositions according to the invention, having favourable homeotherm toxicity, are suitable for controlling pathogenic endoparasites encountered in humans and in animal husbandry and livestock breeding, in productive livestock, breeding stock, zoo animals, laboratory animals, animals used in testing, and pets. They are active against resistant and normally sensitive species and against all or some stages of developments of the pests. By controlling the pathogenic endoparasites, it is intended to reduce disease, mortality and decreasing performance (for example in the production of meat, milk, wool, hides, eggs, honey, etc), so that more economical and simpler animal keeping is possible by using the active compounds. The pathogenic endoparasites include cestodes, trematodes, nematodes and acantocephales:

Praziquantel or epsiprantel controls especially the following endoparasites:

from the order of the Pseudophyllidea, for example: Diphyllobothrium spp., Spirometra spp., Schistocephalus spp., Ligula spp., Bothridium spp., Diphlogonoporus spp.
from the order of the Cyclophyllidea, for example: Mesocestoides spp., Anoplocephala spp., Paranoplocephala spp., Moniezia spp., Thysanosomsa spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.
from the subclass of the Monogenea, for example: Gyrodactylus spp., Dactylogyrus spp., Polystoma spp.
from the subclass of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

Emodepside controls especially the following endoparasites:

from the order of the Enoplida, for example: Trichuris spp., Capillaria spp., Trichomosoides spp., Trichinella spp.
from the order of the Rhabditia, for example: Micronema spp., Strongyloides spp.
from the order of the Strongylida, for example: Stronylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp.
Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.
from the order of the Oxyurida, for example: Oxyuris spp., Enterobius spp., Passalurus spp., Syphacia spp., Aspiculuris spp., Heterakis spp.
from the order of the Ascaridia, for example: Ascaris spp., Toxascaris spp., Toxocara spp., Parascaris spp., Anisakis spp., Ascaridia spp.
from the order of the Spirurida, for example: Gnathostoma spp., Physaloptera spp., Thelazia spp., Gongylonema spp., Habronema spp., Parabronema spp., Draschia spp., Dracunculus spp.
from the order of the Filariida, for example: Stephanofilaria spp., Parafilaria spp., Setaria spp., Loa spp., Dirofilaria spp., Litomosoides spp., Brugia spp., Wuchereria spp., Onchocerca spp.
from the order of the Gigantorhynchida, for example: Filicollis spp., Moniliformis spp., Macracanthorhynchus spp., Prosthenorchis spp.

Using suitable combinations of active compounds, it is possible to cover the entire spectrum of the endoparasites listed above. Particular preference is given to using the compositions according to the invention against: Toxocara cati, Toxascaris leonina, Ancylostoma tubaeforme, Dipylidium caninum, Taenia taeniaeformis and Echinococcus multilocularis.

The livestock and breeding stock include mammals, such as, for example, cattle, horses, sheep, pigs, goats, camels, water buffalo, donkeys, rabbits, fallow deer, reindeer, fur-bearing animals, such as, for example, mink, chinchilla or racoon, birds, such as, for example, chickens, geese, turkeys, ducks, ostriches.

The laboratory and test animals include mice, rats, guinea pigs, golden hamsters, dogs and cats.

The pets include dogs and cats.

Administration can be both prophylactic and therapeutic.

Very particular preference is given to the administration to cats.

The compositions according to the invention are preferably fluids, for example suspensions, emulsions or, in particular, solutions. According to the invention, the solvent used is 1,2-isopropylideneglycerol, which can be used as only solvent or in a mixture with other solvents. Such solvent mixtures preferably comprise at least 60% by volume of 1,2-isopropylideneglycerol. Particular preference is given to using 1,2-isopropylideneglycerol as only solvent.

Surprisingly, it has been found that such compositions according to the invention comprising the solvent 1,2-isopropylideneglycerol—preferably in concentrations of at least 60% by volume—are, when applied externally, particularly effective against nematodes and cestodes, without it being necessary to resort to penetration enhancers as usually recommended in the prior art.

Suitable further solvents for the solvent mixtures mentioned above are pharmaceutically acceptable organic solvents with suitable solubility properties, for example N-methyl-2-pyrrolidone (NMP).

To minimize the risk of the hydrolysis of 1,2-isopropylideneglycerol and to considerably enhance the stability of the formulations, it is recommended to keep the water content of the compositions according to the invention as low as possible; usually, the water content should not exceed 1% by weight and preferably not exceed 0.5% by weight.

One hydrolysis product of 1,2-isopropylideneglycerol is acetone, the concentration of which in medicaments should be kept as low as possible, in accordance with international guidelines. Furthermore, acetone may affect the uptake of the active compounds into the body. The change of these properties, for example by hydrolysis of the 1,2-isopropylideneglycerol during storage, is unacceptable for a medicament.

The solvent content of the compositions according to the invention is usually from 60 to 96% by weight, preferably 70 to 96% by weight, particularly preferably from 80 to 90% by weight, based on the total weight of the finished composition.

It may be advantageous to add further auxiliaries customary in veterinary medicine to the compositions according to the invention.

Preferred auxiliaries are, for example, oxidation stabilizers, such as, for example, butylated hydroxyanisol (BHA), butylated hydroxytoluene (BHT) and ascorbic acid. These may be employed, for example, in concentrations of from 0.1 to 1% by weight, preferably from 0.3 to 0.7% by weight, based on the total weight of the finished composition.

Further preferred auxiliaries are stabilizers, such as, for example, organic acids, in particular lactic acid. These are usually employed in amounts of from 1 to 5% by weight, preferably from 1 to 3% by weight, based on the total weight of the finished composition.

If appropriate, the compositions according to the invention may also comprise synergists or further active compounds.

Ready-to-use preparations usually comprise the active compounds in concentrations of from 0.1 to 25% by weight, preferably 0.1 to 20% by weight, where concentrations of from 0.5 to 5% by weight, in particular from 1 to 3% by weight, are preferred for emodepside and concentrations of from 1 to 15% by weight, in particular from 5 to 10% by weight, are preferred for praziquantel or epsiprantel.

The compositions are prepared by mixing appropriate amounts of the components in suitable vessels; preferably, the components are mixed until a clear solution is formed.

In general, it has been found to be advantageous to meter the compositions according to the invention such that per application from about 1 to about 100 mg of the active compound in question are administered per kg of body weight. Preferred in the case of emodepside are from 1 to 20 mg, in particular from 1 to 10 mg, of active compound per kg of body weight and in the case of praziquantel or epsiprantel from 5 to 50 mg, in particular from 5 to 20 mg, of active compound per kg of body weight.

### EXAMPLE 1

100 g of solution comprising:

```
  7.94 g of praziquantel
  1.98 g of emodepside
87.58 g of 1,2-isopropylideneglycerol
  2.00 g of lactic acid
  0.50 g of butylated hydroxyanisol
```

In clinical studies, the composition according to Example 1 has been found to be highly effective in cats against infections with Toxocara cati, Toxascaris leonina, Ancylostoma tubaeforme, Dipylidium caninum, Taenia taeniaeformis and Echinococcus multiocularis.

1. A composition comprising:
(a) emodepside,
(b) praziquantel or epsiprantel, and,
(c) a solvent mixture, wherein the solvent mixture comprises 1,2-isopropylideneglycerol and wherein the water content of the composition is at most 1% by weight.

2. The composition according to claim 1, characterized in that it comprises emodepside and praziquantel.

3. The composition according to claim 1, wherein the solvent mixture comprises at least 60% by volume 1,2-isopropylideneglycerol.

4. The composition according claim 1, wherein the solvent mixture comprises only 1,2 isopropylideneglycerol.

5. A process for preparing the according to claim 1, characterized in that the active compounds are mixed with the solvent mixture and, if appropriate, further auxiliaries.

6.-7. (canceled)

8. The composition of claim 1, comprising:
a. from about 1-15% by weight emodepside,
b. from about 5-10% by weight praziquantel or epsiprantel, and,
c. from about 80-90% by weight solvent mixture of the composition.

9. A method of controlling endoparasites comprising applying to an animal in need thereof an effective amount of the composition of claim 1.

10. The method of claim 8, wherein the animal is a cat.

11. The method of claim 9, wherein the endoparasites are selected from the group consisting of Toxocara cati, Toxascaris leonina, Ancylostoma tubaeforme, Dipylidium caninum, Taenia taeniaeformis, Echinococcus multiocularis, and combinations thereof.
